(19) United States
(12) Patent Application Publication
(10) Pub. No.: US 2004/0121304 A1
(43) Pub. Date: Jun. 24, 2004

(54) Method for screening for progesterone receptor isoform-specific ligands and for tissue-selective progesterone receptor ligands

(76) Inventors: Ulrike Fuhrmann, Berlin (DE); Christia Hegele-Hartung, Mulheim a d Ruhr (DE); Michael Klotzbucher, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/250,400

(22) PCT Filed: Dec. 21, 2001
(86) PCT No.: PCT/EP01/15200

(51) Int. Cl. C12Q 1/00; G01N 33/53; G01N 33/567; C12N 15/86

(52) U.S. Cl. 435/4; 435/456; 435/7.2

(57) ABSTRACT

The present invention provides a method for screening for progesterone receptor isoform specific ligands as well as a first method for screening for tissue-selective progesterone receptor ligands, both methods comprising selecting progesterone receptor isoform A or progesterone receptor isoform B selective ligands by means of an assay involving cells stably transfected with plasmids expressing the progesterone receptor isoform A or B. Furthermore, the present invention provides a second method for screening for tissue-selective progesterone receptor ligands, comprising in vivo tests in desired target tissues. The present invention further relates to cell lines suitable for this transactivation assay, a respective assay kit and medical uses of the isoform-specific and/or tissue-selective progesterone receptor ligands according to the present invention.

### FIELD OF THE INVENTION

The present invention relates to a method for screening for progesterone receptor ligands having a selectivity for either the progesterone receptor isoform A or the progesterone receptor isoform B. The present invention further relates to methods for screening for tissue-selective, in particular breast/uterus-selective, progesterone receptor ligands. The present invention further relates to an assay kit for screening for progesterone receptor isoform specific ligands. Furthermore, the present invention relates to SK-N-MC cell lines stably transfected with a plasmid expressing the progesterone receptor isoform A or the progesterone receptor isoform B and a progesterone-inducible luciferase reporter gene. Finally, the present invention also relates to the progesterone receptor isoform specific ligands and to the tissue-selective progesterone receptor ligands identified through the methods of the present invention. The present invention also relates to medical uses of the progesterone receptor isoform specific and the tissue-selective progesterone receptor ligands identified through the methods of the present invention.

### BACKGROUND OF THE INVENTION

Progesterone is a unique reproductive hormone, and it plays a decisive role for tissues of female reproduction. Its principal target organs are uterus, breast, ovary and the hypothalamus-pituitary axis. The development of new progestins and the understanding of their mode of action have been part of one of the greatest chemical and biological research efforts on a single group of substances (steroids). In addition to the primary use as birth control for women (e.g., oral contraception (OC)), progestins, combined with estrogen, are widely used in hormone replacement therapy (HRT). Progestins are also used to treat several gynecological disorders, e.g., dysmenorrhea, endometriosis, and dysfunctional uterine bleeding caused by hormonal deficiency or imbalance. Due to undesirable side effects or cross-reactivities with other receptors, the development of new generations of progestins to improve their selectivity profile has been a great challenge. Additionally, the exploration of therapeutic applications such as oncology demands progestins with new activity profiles.

Studies revealed that several nonsteroidal progestins, that are members of the class of 5-aryl-1,2-dihydro-5H-chromeno[3,4-f]quinolines, display certain tissue-selective progestational effects (L. Zhi et al., J. Med. Chem. 1998, 41, 291-302). In particular, it was observed that in two in vivo rodent models of progestational activity, a pregnancy maintenance assay and a uterine wet weight assay, two members of said class of compounds displayed potent progesterone-like effects. In a third model for progestational activity, the mammary end bud assay, these compounds were significantly less active (J. P. Edwards et al., J. Med. Chem. 1998, 41, 2779-2785).

The provision of a dissociated progestin with e.g. antiproliferative potential in the breast tissue and, at the same time, beneficial effects in the endometrium seems especially desirable, as there is a large number of epidemiological studies about the relation between breast cancer incidence and use of combined oral contraceptives (COCs) and HRT, especially with respect to extended periods of use (see e.g., K. E. Malone et al., Epidemiologic Reviews 1993, 15, 80-97 and Standford et al., Epidemiologic Reviews 1993, 15, 98-107). Although the risks are contradictory and controversial, there is clear evidence that many years of intake might enhance the mitotic activity of normal breast epithelial cells. These data give the implication that endogenous and exogenous progestins might be a potentially hazardous stimulus to the breast tissue (A. Brzezinski et al., Gynecol. Endocrinol. 1997, 11, 357-364). Recent studies confirmed again that there is strong evidence that the addition of a progestin to HRT enhances markedly the risk of breast cancer relative to estrogen use alone (R. K. Ross et al., J. Natl. Cancer Inst. 2000, 16, 92(4), 328-332; see also: C. Schairer et al., JAMA 2000, 283(4), 485-491). Thus, there exists a strong need for new, selective progesterone receptor ligands for innovative fertility control and HRT as well as a method and kit for identifying such selective ligands.

Like other steroid hormone receptors, PR is expressed in two isoforms in certain organisms, including humans (H. Gronemeijer et al., J. Steroid Biochem. 1991, 40, 271-278; T. T. Ilenchuck et al., Pndocrinology 1987, 120, 1449-1456). Thus, also in human breast tumor and endometrium cell lines two PR isoforms, hPR-A and hPR-B, have been identified (P. Estes et al., Biochemistry 1987, 26, 6250-6262; L. Klein-Hitpass et al., Nucleic Acid Res. 1991, 19, 1227-1234; K. Christensen et al, Mol. Endocrinol. 1990, 24, 1465-1473). Human PR-A is a truncated form of hPR-B and lacks 164 amino acids at the N-terminus (K. B. Horwitz et al., Endocrinology 1983, 113, 2195-2201). Both isoforms are identical in the DNA-binding and ligand-binding domain and induce progestin-mediated gene transcription. However, they have a different transactivation behavior as outlined below.

Potentially, due to the difference in the length of the N-termini of the PR-A and PR-B isoforms, the identical ligand bound to PR-A or PR-B could induce different conformations of the two isoforms, resulting in interactions with different sets of coactivators/corepressors, which in turn could be the reason for the different transactivation behavior observed. Therefore, it should be possible to identify compounds, which selectively activate one isoform by inducing an interaction with the receptor and coactivators and selectively inhibit the other isoform by inducing interaction of the receptor and corepressors. Support for this hypothesis can be taken from a recent publication of Tetel et al. (Mol. Endocrinol. 1999, 13, 910-924), reporting a hormone dependent intramolecular interaction between the amino and the carboxy terminal domains of PR.

Both PR-isoforms are expressed in all progesterone target organs tested so far (e.g. breast, uterus). However, there is strong evidence that PR-A and PR-B function in a tissue-specific manner to mediate responses to progesterone. Isoform-specific knock-out mice show different functions of PR-A and PR-B in the sane target organ. Based on these studies, PR-B seems to be the most responsible receptor for mammary gland proliferation and differentiation, whereas the antiproliferative action of progestins on the uterine epithelium and on ovulation is most likely mediated by PR-A (B. Mulac-Jericevic, Science 2000, 289, 1751-1754; Orla Conneely, Endocrine Society Meeting, Toronto, June 2000).

Despite the potential desirability of identifying PR isoform specific ligands, the prior art fails to disclose any method used specifically to identify such ligands. The prior art furthermore fails to provide a method for identifying tissue-selective PR ligands. Accordingly, the prior art does not provide PR isoform-specific and/or tissue-selective PR ligands.

General assays and methods for determining the transcriptional activity of an intracellular receptor when exposed to a ligand have been developed. For example, U.S. Pat. No. 5,071,773 describes an assay by which hormone intracellular receptors, ligands for these receptors and proteins having transcriptional activating properties of a hormone intracellular receptor can be identified.

A hormone-dependent transcription modulation system was established on the basis of a two-step transfection procedure of the human PR-A and PR-B isoforms and a progesterone receptor-responsive reporter (R. Dijkema et al., J. Steroid. Biochem. Molec. Biol. 1998, 64, 147-156). It was shown that the Chinese Hamster Ovary (CHO) cell lines expressing hPR-isoforms and PR-responsive reporter used in this study can provide an efficient and accurate tool for the quantitative determination of (anti)progestins in vitro. However, all progestagenic agonists and antagonists tested were active towards the individual PR-isoforms in a similar, if not identical way. Thus, no selection of PR isoform specific compounds was performed so far.

The obtained transactivation data were compared to relative binding affinities and biopotency estimations with McPhail staging of rabbit endometrium, ovulation and pregnancy interruption in rats (W. G. E. J. Schoonen et al, J. Steroid Biochem. Molec. Biol. 1998, 64, 157-170). It was found that due to the good resemblance of these in vitro and in vivo tests, direct pre-screening of agonists, antagonists and partial antagonists of PR in general is possible. Thus, this transactivation assay can replace binding assays and is a valuable pre-screening tool for in vivo studies involving a particular class of chemical compounds.

The present invention is based on the new theory that isoform-specific ligands of PR activity may allow tissue-selective modulation of progestin activity in hormonal therapy. Therefore, one approach towards mammary tissue protective PR ligands which, however, maintain their desired effects on other target organs, such as the uterus, could be to identify pure agonists that selectively stimulate PR-A transactivation or pure antagonists that selectively inhibit PR-B transactivation or partial agonists with stronger agonistic activity on PR-A and stronger antagonistic activity on PR-B. However, the present invention is not intended to be limited to this approach. Furthermore, this approach should be applicable to other tissue systems as well. Although many progestins are on the market, no progestin with dissociated antiproliferative potential, preferably on breast tissue, is presently available.

### OBJECTS OF THE INVENTION

As outlined above, it is desirable to design progesterone receptor (PR) ligands with target tissue specificity, i.e. to achieve, for example, dissociation of progestational activity in the reproductive tract from that in the breast and thereby reduce undesired effects of COC's and HRT, such as an increased risk of breast cancer incidence and thus protect both the mammary gland and the uterus. Furthermore, it is desirable to identify PR ligands having a selectivity for one of the PR isoforms, PR isoform A or PR isoform B. Thus, a need exists for a method for screening for PR ligands exhibiting a certain desired tissue-selectivity.

It is a further object of the present invention to provide a method for screening for PR isoform specific ligands, i.e. PR ligands having a selectivity for the PR isoform A or B. Preferably, the method for identifying PR isoform specific ligands should be suitable for identifying ligands that are pure agonists that selectively stimulate PR-A transactivation or pure antagonists that selectively inhibit PR-B transactivation or partial agonists with stronger agonistic activity on PR-A and stronger antagonistic activity on PR-B.

Furthermore, it is an object of the present invention to provide a method for screening for tissue-selective PR ligands for application as a medicament, e.g., in fertility control and HRT. Preferably, the desired method for screening for tissue-selective PR ligands should be suitable for screening for PR ligands that exhibit an inhibiting or at least a non-stimulating effect on a first selected target tissue, preferably breast tissue, and a protective effect on a second selected target tissue, preferably uterine tissue.

Desirably, the screening methods should be relatively simple, highly selective and efficient, thus possibly allowing high-throughput screening. Furthermore, the screening methods should be applicable for PR pure agonists, pure antagonists and partial agonists/antagonists.

It is a further object of the present invention to provide an assay kit suitable for performing the method for screening for isoform-specific PR ligands in vitro.

It is another object of the present invention to provide at least one type of cell line that is a preferred cell line for use in a method for screening for isoform-specific and/or tissue-selective PR ligands and that is preferably comprised in the assay kit.

It is yet another object of the present invention to provide PR isoform A or B specific ligands and/or tissue-selective, preferably breast/uterus selective, PR ligands. These PR isoform A or B specific ligands and/or tissue-selective PR ligands should be suitable for use as medicaments, preferably in fertility control and HRT.

Finally, it is an object of the present invention to provide a method for selectively inhibiting or stimulating PR isoform A or B. And it is another object of the present invention to provide a method for selectively modulating PR mediated conditions in a first selected tissue with respect to a second selected tissue, with the first selected tissue preferably being breast tissue and the second selected tissue preferably being uterine tissue.

All these objects are surprisingly achieved by the provision of the screening methods for PR isoform specific ligands and/or for tissue-selective PR ligands, the assay kit, the SK-N-MC cell lines, the PR isoform A or B specific and/or the tissue-selective PR ligands, the use of the respective ligands as medicaments and the method for selectively inhibiting the PR-A or PR-B isoform as well as the method for selectively modulating PR mediated effects in a selected tissue according to the present invention as expressed in the claims.

### SUMMARY OF THE INVENTION

The present invention provides a method for screening for PR isoform A or B specific ligands as well as a first and a second method for screening for tissue-selective PR ligands. Both the method for screening for PR isoform-specific ligands as well as the first method for screening for tissue-selective PR ligands according to the present invention basically comprise selecting PR ligands having a selectivity for either the PR-A or the PR-B isoform. The first method for screening for tissue-selective PR ligands additionally comprises subjecting the PR isoform-specific ligands identified by the PR isoform specific in vitro test according to the present invention to in vivo tests in a first and second target tissue and selecting those ligands having the desired activity with respect to said first and said second target tissue. The second method for screening for tissue-selective PR ligands comprises subjecting PR ligands to in vivo tests in the desired target tissues and selecting those ligands that selectively modulate PR mediated effects in one of the desired target tissues with respect to the other desired target tissue.

Thus, the first method for screening for tissue-selective PR ligands according to the present invention is based on the new and surprising concept that certain steroidal or non-steroidal PR ligands, which are selective PR-A or PR-B-specific pure agonists, pure antagonists or partial agonists/antagonists as evidenced by the selectivity assay, preferably a transactivation assay according to the present invention, induce tissue-selective progestational effects.

The second method for screening for tissue-selective PR ligands according to the present invention is based on the different progesterone receptor mediated effects induced by tissue-selective PR ligands in different target organs as identified through in vivo tests.

The present invention also provides PR isoform A or B specific ligands and/or tissue-selective PR ligands that are identified through the methods according to the present invention. The PR isoform A or B specific and/or tissue-selective PR ligands according to the invention are suitable for use as medicaments, e.g. in fertility control or HRT. Preferably, the PR isoform specific ligands identified by the method according to the present invention selectively block or at least do not affect PR-B transactivation, but are still strong agonists for PR-A. The tissue-selective PR ligands identified by the first and/or second method for screening for tissue-selective PR ligands according to the present invention preferably inhibit differentiation and proliferation of the mammary epithelium, whereas the progestational activity in the reproductive tract is maintained or even enhanced. The tissue-selective PR ligands according to the present invention thus exert a protective effect both on the mammary gland as well as the uterus.

In another aspect, the present invention provides an assay kit suitable for performing the method according to the present invention, i.e. for screening for PR-A or PR-B isoform-specific ligands.

Furthermore, the present invention provides SK-N-MC cell lines stably transfected with either a plasmid expressing PR-A or PR-B. These cell lines are especially suitable for use in the screening method for PR isoform-specific ligands as well as the first method for screening for tissue-selective PR ligands according to the present invention and as part of the assay kit according to the present invention.

In yet another aspect, the present invention provides a method for selectively inhibiting or stimulating PR isoform A or PR isoform B mediated effects. The present invention also provides methods for selectively modulating PR-mediated conditions in a selected tissue, preferably for not influencing or inhibiting PR mediated, preferably PR-B mediated, conditions in a first selected tissue, preferably breast tissue, and, at the same time, enhancing PR mediated, preferably PR-A mediated, effects in a second selected tissue, preferably uterine tissue.

### DETAILED DESCRIPTION OF THE INVENTION

In a first aspect, corresponding to claims 1 to 9, the present invention pertains to a method for screening for progesterone receptor (PR) isoform A or B specific ligands. Basically, the method comprises the steps of:

(a) stably transfecting first cells with a plasmid expressing the progesterone receptor isoform A;

(b) stably transfecting second cells with a plasmid expressing the progesterone receptor isoform B;

(c) further stably transfecting said first and said second cells with a plasmid comprising a reporter gene linked to a hormonally responsive promoter;

(d) contacting said first and said second cells with a ligand to be tested;

(e) determining the transcription efficacy and/or potency of said reporter gene in said first and second cells; and

(f) selecting a ligand having a selectivity for the progesterone receptor isoform A or the progesterone receptor isoform B.

In a second aspect, corresponding to claims 10 to 20, the present invention pertains to a first method for identifying tissue-selective progesterone receptor ligands. Basically, this method comprises the selection of isoform specific progesterone receptor ligands, i.e. ligands having a selectivity for either the progesterone receptor isoform A (PR-A) or the progesterone receptor isoform B (PR-B) according to the first aspect of the present invention as explained above. Additionally, the first method for identifying tissue-selective PR ligands comprises subjecting the selected PR isoform-specific ligands to in vivo tests in a first and second tissue and selecting those ligands having the desired acitivity in said first and second tissue. Thus, the fist method for identifying tissue-selective PR ligands comprises at least the following steps:

(a) stably transfecting first cells with a plasmid expressing the progesterone receptor isoform A;

(b) stably transfecting second cells with a plasmid expressing the progesterone receptor isoform B;

(c) further stably transfecting said first and said second cells with a plasmid comprising a reporter gene linked to a hormonally responsive promoter;

(d) contacting said first and said second cells with a ligand to be tested;

(e) determining the transcription efficacy and/or potency of said reporter gene in said first and second cells;

(f) selecting a ligand having a selectivity for the progesterone receptor isoform A or the progesterone receptor isoform B;

(g) subjecting the ligand selected in step (f) to in vivo tests in a first and a second target tissue; and

(h) selecting the ligand having the desired activity with respect to said first and said second target tissue.

In the present invention, the “selectivity” for one PR isoform, PR-A or PR-B as determined in step (f) of both methods as explained above, i.e. the method for screening for PR isoform-specific ligands and the first method for screening for tissue-selective PR ligands according to the present invention, is defined as a difference in transcription efficacy induced by a certain test ligand in said first and said second cells. Preferably, this difference must be above or equal to 10%, more preferably above or equal 15% and most preferably even higher, such as above or equal 20%.

“Transcription efficacy” in the context of the present invention is defined as the response achieved with a defined concentration of test ligand relative to a standard in either PR-A or PR-B transfected cells.

Another potential parameter for evaluating the activity and thus also the selectivity of a test ligand with respect to either PR-A or PR-B is “potency”, i.e. the EC₅₀ (for agonism) or IC₅₀ (for antagonism) value, determined in vitro in either PR-A or PR-B transfected cells. Thus, “selectivity” for one PR isoform, PR-A or PR-B as determined in step (f) of both above methods, may also—additionally or alternatively—be defined as a difference in potency achieved by a certain test ligand in said first cells with respect to said second cells. Preferably, this potency difference should be in the range of or above a factor of 10, more preferably even larger.

Thus, the “selectivity” defined in step (f) of the method for screening for PR isoform-specific ligands as well as the first method for screening for tissue-selective PR ligands according to the present invention is defined such that the difference in transcription efficacy determined for a potential PR isoform-specific and/or tissue-selective PR ligand in said first and said second cells should preferably be above or equal to 10%, more preferably above or equal to 15% and most preferably above or equal to 20% and/or the potency achieved with said ligand in said first cells should preferably differ by a factor of at least 10, more preferably by an even larger factor, from the potency achieved with said ligand in said second cells. Thus, both efficacy and potency may be used, either alternatively or in combination, as criterion for determining PR-A/PR-B selectivity according to step (f) in both above-described screening methods.

In a third aspect, corresponding to claims 69 to 73, the present invention pertains to a second method for identifying tissue-selective PR ligands. This second method is based on in vivo tests in different selected target tissues and provides PR ligands that selectively modulate PR mediated effects in these target tissues. Thus, the second method for screening for tissue-selective PR ligands according to the present invention comprises the steps of

(a) subjecting a progesterone receptor ligand to at least one in vivo test in a first selected tissue,

(b) subjecting the same progesterone receptor ligand to at least one in vivo test in a second selected tissue, and

(c) selecting a progesterone receptor ligand that selectively modulates progesterone receptor mediated effects in said first selected tissue with respect to said second selected tissue.

The term “ligand” in the present invention is meant to encompass any compound binding to a receptor (in the present invention, preferably the progesterone receptor) and inducing either activation or inhibition or any other potential form of interaction with this receptor. The term “ligand” is also used for compounds which are potential ligands of a receptor, but whose binding affinity and/or activity at the receptor is not yet known, but has to be tested with methods according to the present invention.

The term “plasmid” in the present invention is intended to encompass “plasmid vectors”.

Thus, one achievement of the present invention is that for the first time a correlation of PR-A/PR-B isoform specificity of PR ligands with tissue-selectivity of PR ligands, in particular breast/uterus selectivity, is possible. Furthermore, it has become possible by the methods of the present invention to identify tissue-selective, in particular breast/uterus-selective, PR ligands. It is particularly advantageous to design tissue-selective progestins for use in fertility control and hormone (replacement) therapy in order to avoid disadvantages of known therapies, for example an increased incidence of breast cancer. Isoform-specific and/or tissue-selective PR ligands identified through the methods according to the present invention only activate the progesterone receptor at a specific target tissue, but not at any other, undesired tissue, thus rendering these treatments well tolerable and less prone to serious side effects or even the induction of further health problems. Furthermore, these progestins may be administered in a much smaller dose due to their target specificity than the progestins currently used in (combined) oral contraceptives and HRT. Moreover, the present invention allows for tailored modulation of PR mediated conditions by the use of PR isoform-specific and/or tissue-selective PR ligands and may thus open up a path for further beneficial uses of progestins, in particular progesterone receptor pure agonists, pure antagonists and partial agonists/antagonists, in the treatment of PR related conditions. Isoform-specific and/or tissue-specific PR ligands identified by the method according to the present invention will even contribute to the further elucidation of biological pathways involved with certain pathological conditions related with intracellular receptors, e.g. progesterone receptors in general and PR isoforms in particular. However, the methods according to the present invention for screening for PR isoform-specific and first method according to the present invention for screening for tissue-selective PR ligands may be applied mutatis mutandis to other receptor/ligand systems involving different forms of a receptor and/or different receptor mediated effects in selected target organs (e.g. the androgen receptor system). Furthermore, both methods according to the present invention for screening for tissue-selective PR ligands may also be applied mutatis mutandis to other desired tissues or target organs.

In the following, the methods according to the present invention for screening for PR isoform specific and/or tissue-selective PR ligands will be described in more detail. As steps (a) to (f) are identical in their broadest as well as their preferred meanings in the method for screening for PR isoform-specific ligands and in the first method for screening for tissue-selective PR ligands, these steps (a) to (f) will be described in detail only with reference to the method for screening for PR isoform-specific ligands. The additional steps (g) and (h) comprised in the first method for screening for tissue-selective PR ligands are explained in detail afterwards.

The method for screening for PR isoform specific ligands according to the first aspect of the present invention is preferably a transactivation assay, comprising a multiple stable transfection, i.e. steps (a) to (c) as defined above. Preferably, the cells used in this screening method according to the first aspect of the present invention as well as comprised in the assay kit according to the present invention (which will be described herein below) are SK-N-MC cells (human neuroblastoma cells). This cell line is especially well suited for the purposes of the present invention, as it comprises monospecific cells, i.e. cells expressing only PR, but no other hormonal receptors, e.g. androgen, glucocorticoid or other steroid receptors. Furthermore, in these specific cells, the PR isoform A is equally active as the PR isoform B. This is a clear advantage of the new and inventive cells according to the present invention and which will be described in more detail below. However, for the screening methods according to first and second aspect of the present invention as well as for the assay kit according to the present invention, other cell lines are equally suitable, for example, the Chinese Hamster Ovary (CHO) cells or the CV-1 cells.

In the method for screening for PR isoform A or B specific ligands according to the present invention, cells as described above are stably transfected with a plasmid expressing PR-A or PR-B, respectively (a detailed description of an exemplary method for obtaining the stably transfected cells according to the present invention is given in Example 5). In other words, “first cells” are obtained specifically expressing PR-A (corresponding to step (a)) and “second cells” specifically expressing PR-B (corresponding to step (b)). The most preferred cells used in the screening methods according to the first and second aspect of the present invention and the assay kit according to the present invention are SK-N-MC cells. For the transactivation assay, it is most preferred that cells are stably transfected with a plasmid expressing human PR-A or PR-B (hPR-A or hPR-B).

The plasmids expressing PR-A or PR-B, respectively, are preferably plasmids pRSVhPR-neo (the PR-B plasmid; for the cells specifically expressing PR-B) as well as phPR-2 (the PR-A plasmid) and pRSV-neo (for the cells specifically expressing PR-A), respectively. Example 5 gives a detailed description of how and/or where to obtain these plasmids. They contain the full-length coding sequence of either PR-A or PR-B expressed from the long terminal repeat (LTR) of the rous sarcoma virus (RSV). The neomycin resistance gene, expressed from the SV40 early promoter, allows for neomycin selection of stable transfectants. However, other constructs are also envisaged, e.g., comprising any other antibiotic resistance gene suitable for the purposes of the present invention.

Furthermore, the first and second cells according to the present invention and suitable for use in the screening methods of the first and second aspect of the present invention and as part of the assay kit according to the present invention (preferably the SK-N-MC cells as described above) are further stably transfected in step (c) with a plasmid comprising a reporter gene linked to a hormonally responsive promoter. Preferably, this plasmid is a pMTV-LUC plasmid, wherein the hormonally responsive promoter is the mouse mammary tumor virus (MTV) promoter, which is linked to the luciferase (LUC) reporter gene (for a detailed description, see again Example 5). However, also other promoter/reporter gene systems suitable for the purpose of the present invention are envisaged. Furthermore, the cells stably transfected with the PR-A or PR-B plasmids and the promoter/reporter gene are additionally stably transfected as described e.g. in Example 5 with another antibiotic resistance gene, preferably a puromycin resistance gene (e.g., pSV2pac). As above, any other antibiotic resistance gene suitable for the purposes of the present invention may be used as well. Finally, the antibiotic resistant SK-N-MC clones stably expressing PR-A and MTV-LUC or PR-B and MTV-LUC (or, instead of MTV-LUC, any other suitable promoter/reporter gene system, as explained above) were selected for transactivation studies, i.e. for the method for screening for PR isoform-specific ligands as well as for the first method for screening for tissue-selective PR ligands according to the present invention.

In step (d) of the method for screening for PR-A or PR-B isoform-selective ligands according to the present invention, said first and said second stably transfected cells (i.e. PR-A or PR-B expressing cells, respectively) are contacted with a ligand to be tested, i.e. a compound being a potential PR isoform-specific ligand. Ligands to be tested can be of steroidal or non-steroidal nature. The method can be performed with compounds having already been tested on PR affinity/activity in general and found to bind to and activate or inhibit PR, but also with compounds of which their PR ligand potential is unknown. In case the compound to be tested has already qualified as PR ligand, it can be a pure agonist, pure antagonist or partial agonist/antagonist of PR.

After said first and said second stably transfected cells have been incubated in step (d) with a ligand to be tested, the transcription efficacy and/or potency of the potential PR-A or PR-B selective ligand in said first and said second cells is determined in step (e) of the screening method according to the present invention. The efficacy and/or the potency of said potential PR isoform-specific ligand as defined above can be determined via the level of expressed reporter gene product (cf. Example 1). In case the reporter gene is a LUC reporter gene as described above as a preferred embodiment of the present invention, the level of LUC reporter gene product is determined in cell lysates and is measured as RLU (relative light units). However, any other method for determining the transcription efficacy and/or potency in said first and second cells is equally applicable in the screening methods as well as in the assay kit according to the present invention.

In particular, to determine agonistic activity of the ligand in said first or said second cells, prior to the luciferase assay (in case the reporter gene is a LUC reporter gene as described above) or to any other assay suitable for the purposes of the present invention, the respective cells (PR-A and PR-B expressing cells, but each group separately) are cultured in the presence of increasing concentrations of the ligand to be tested, preferably 0.01 nmol/l to 1 μmol/l. As a positive control for reporter gene induction, the same type of cells is treated with a standard progestin, preferably the synthetic progestin R5020 (promegestone), preferably with concentrations of 0.01 mmol/l to 1 μmol/l. As a negative control for reporter gene induction, the same type of cells are preferably cultured in 1% ethanol. For the determination of agonistic efficacy, all obtained data points are calculated relative to a standard progestin, preferably relative to the induction of LUC activity obtained by treating the cells with 10⁻⁷ mmol/l of R5020. For the determination of agonistic potency [nM], the EC₅₀ value (i.e., the amount [nM] of ligand which provides for half of the maximum agonistic effect of this ligand) is determined graphically (see, e.g., Example 1).

For the determination of antagonistic activity, the respective cells (PR-A and PR-B expressing cells, but each group separately) are treated with a standard progestin, preferably with 0.1 nmol/l R5020 and additionally with increasing amounts of the ligand to be tested, preferably 0.01 mmol/l to 1 μmol/l. As a positive control for the inhibition of reporter gene transcription the same type of cells is cultured in increasing amounts of a standard antiprogestin, preferably the known antiprogestin mifepristone, preferably in concentrations of 1 pmol/l to 100 nmol/l. For the determination of antagonistic efficacy, all obtained data points are calculated relative to a standard progestin, preferably relative to the induction of LUC activity, preferably obtained by treating the cells with 0.1 nmol/l of R5020. For the determination of antagonistic potency [nM], the IC₅₀ value (i.e., the amount [nM] of ligand which provides for half of the maximum antagonistic effect of this ligand) is determined graphically (see, e.g., Example 1).

For said first and said second cells, i.e. the PR-A specific cells and the PR-B specific cells, a certain level of expressed reporter gene product is obtained for the ligand to be tested and thus a certain transcription efficacy and/or potency of the reporter gene in said first and said second cells induced by said ligand can be determined. According to step (f) of the screening method according to the first and second aspect of the present invention, ligands having a selectivity for either PR-A or PR-B as defined above have to be selected. Preferably, for the purposes of the present invention, ligands having purely PR-A agonistic or purely PR-B antagonistic activity or ligands having stronger partial agonistic activity on PR-A and stronger partial antagonistic activity on PR-B are selected. Other exemplary ligands to be selected by these methods of the present invention are ligands having purely PR-A antagonistic and purely PR-B agonistic activity or ligands having partial PR-A antagonistic/PR-B agonistic activity. Thus, as defined above, the difference in transcription efficacy induced by a ligand tested in both groups of PR isoform-specific cells, i.e. in said first and said second cells, has to be preferably above or equal to 10%, more preferably above or equal to 15% and most preferably even higher, such as above or equal to 20% and/or the potency achieved with such ligand in said first cells should preferably differ by a factor of at least 10 from the potency achieved with such ligand in said second cells.

One preferred embodiment of the present invention is e.g. the assay according to Example 1, wherein a detailed description of an exemplary method for screening for PR isoform-specific ligands according to the invention is given. In Example I, some exemplary results obtained for some PR-A or PR-B isoform-specific ligands identified through the method according to the invention as defined above are summarized. Additionally, in Example 5 a detailed description of the stable transfections is provided.

As explained above, the first method for screening for tissue-selective PR ligands according to the second aspect of the present invention comprises identical steps (a) to (f) as the method for screening for PR isoform-specific ligands according to the present invention as described above. Thus, steps (a) to (f) of the first method for screening for tissue-selective PR ligands (which is the second aspect of the present invention) are disclosed above with respect to the first aspect of the present invention, i.e. the method for screening for PR isoform specific ligands. However, the first method for screening for tissue-selective PR ligands according to the second aspect of the present invention additionally comprises steps (g) and (h), which will be explained below.

According to the new and surprising concept of PR isoform-specificity and tissue-selectivity, once a ligand having the desired selectivity as defined above for PR-A or PR-B has been selected in step (f) of the first method for screening for tissue-selective PR ligands, this ligand is subjected in step (g) of this method to in vivo tests in a first and a second selected tissue, preferably in breast and uterine tissue. These in vivo tests are preferably tests to determine PR mediated effects in said first and said second selected tissue. For example, possible in vivo tests may be those tests described in Examples 2, 3 and 4 of the present invention, i.e. a rodent bioassay on proliferation/differentiation of mammary tissue, a pregnancy maintenance test or an endometrium proliferation/differentiation test in rodents, and an ovulation inhibition test or a superovulation test in rodents. However, any other PR mediated effect-determining in vivo test in the selected first and second tissue is equally suitable for the purposes of the present invention and may be chosen according to the nature of the selected tissues and the desired tissue selectivity profile.

After the in vivo test, again a selection step (step (h)) is performed in the first method for screening for tissue-selective PR ligands according to the second aspect of the present invention, wherein the ligand having the desired activity with respect to the first and second target tissue is selected. Thus, e.g., in one preferred embodiment of the present invention, “desired activity” means that a ligand is selected that does not influence or even inhibit PR-mediated effects in the first target tissue, preferably breast tissue, and enhances or maintains at the same time PR-mediated effects in the second target tissue, preferably uterine tissue. For example, a ligand is selected that does not stimulate or that even inhibits differentiation/proliferation in the mammary tissue, but maintains or enhances its beneficial (protective) effects on the reproductive tract, i.e. exerts antiproliferative activity on the uterus. Such ligands seem especially suitable for use in fertility control, e.g. as oral contraceptives, and hormone replacement therapy (HRT). However, for a person skilled in the art it is evident that the suitability of the present invention is not restricted to the areas of fertility control or HRT, but that the invention can beneficially be applied in other areas involving receptor-mediated conditions as well. Equally, the subject matter of the invention is not limited to PR, but can also be used in other receptor/ligand systems. Furthermore, the present invention may also be extended to more than two target tissues.

According to the third aspect of the present invention, i.e. the second method for screening for tissue-selective PR ligands, in step (a) of this method a PR ligand has to be subjected to at least one in vivo test, preferably at least one in vivo test that is directed to determining PR mediated effects, in a first selected tissue. Preferably, the first selected tissue is breast or uterine tissue, but can also be any other tissue of interest wherein PR mediated effects are observed. Suitable in vivo tests for the purposes of this third aspect of the present invention are e.g. the in vivo tests described in Examples 2, 3 and 4, such as a rodent bioassay on proliferation/differentiation of mammary tissue, a pregnancy maintenance test or an endometrium proliferation/differentiation test in rodents, and an ovulation inhibition test or a superovulation test in rodents. It is finally noted that the second method for screening for tissue-selective PR ligands according to the present invention may analogously be performed for any other receptor ligands and may comprise in vivo tests in any other desired target tissue. It is within the purview of an average skilled person to perform the necessary and appropriate variations to transfer the method as described herein to other receptor systems and/or target tissues.

According to step (b) of the second method for screening for tissue-selective PR ligands according to the present invention, the PR ligands already tested in vivo in a first selected tissue as described above are subjected to at least another in vivo test in a second selected tissue. Preferably, again the in vivo test should be a test suitable for determining PR mediated effects. Such suitable tests are described above with respect to step (a) for the first selected tissue. The second selected tissue according to step (b) is again preferably breast or uterine tissue, whichever of these has not been tested in step (a).

After the in vivo tests in the first and second selected tissue, in step (c) of the second method for screening for tissue-selective PR ligands according to the third aspect of the present invention, the PR mediated effects induced by the tested ligands in said first and said second tissue are compared and those PR ligands are elected that selectively modulate PR mediated effects in said first selected tissue with respect to said second selected tissue.

“Selective modulation” of PR mediated effects for the purposes of this aspect of the present invention means that the selected ligand achieves in the first tissue one or more effect(s) which is/are of a different kind (i.e., agonistic, antagonistic or, in other words, inhibiting, stimulating or not affecting) and/or of a different intensity (i.e., weaker or stronger, or even pertaining longer or shorter) compared to the effect(s) induced by said ligand in the second desired tissue.

Preferably, the selected ligand inhibits or does not influence PR mediated effects in said first tissue, preferably breast tissue, and enhances or maintains PR mediated effects in said second tissue, preferably uterine tissue. Most preferably, the selected ligand inhibits or at least does not influence proliferation/differentiation of the mammary epithelium while it maintains beneficial (i.e., protective) PR mediated effects in the reproductive tract (cf. Examples 2, 3 and 4), e.g. exerts an antiproliferative effect on uterine tissue.

For determining whether a certain test ligand actually selectively modulates a defined PR mediated effect in a defined target tissue with respect to another target tissue, the type of the effect induced by said ligand (i.e., whether the ligand inhibits, does not influence, enhances or maintains the PR mediated effect) as well as the intensity of the induced effect are measured, preferably relative to the effect induced by a known “standard” PR ligand, such as the standard progestin R5020 (promegestone). Then the effects achieved in the first and second target tissue with a certain test ligand are compared and evaluated, preferably under consideration of the desired medical indication (e.g., in HRT etc.). Finally, those PR test ligands are selected that exhibit the desired selectivity for one target tissue with respect to the other target tissue.

It is certainly within the skilled person's knowledge to select and perform suitable in vivo tests as defined above in certain desired target tissues other than the preferred tissues as defined above and to determine the effect induced by a certain test ligand in relation to a suitable standard ligand.

It is also envisaged within the present invention that all methods described above (i.e., methods for screening for PR isoform-specific and/or tissue-selective PR ligands) are applicable for automated high-throughput screening of a large number of potentially PR isoform-specific and/or tissue-selective PR ligands. It is within the skilled person's knowledge to design automated processes based on the methods according to the present invention suitable for high-throughput screening.

Another aspect of the present invention is the provision of PR-A or PR-B specific ligands as well as tissue-selective PR ligands, identified through the screening methods according to the first, second or third aspect of the present invention. As mentioned above, the isoform-specific PR ligands identified through the method according to the first aspect of the present invention can be pure agonists, pure antagonists or partial agonists/antagonists for PR-A or PR-B. Preferably, the isoform-specific PR ligands identified by the method according to the present invention are selective agonists for PR-A and selective antagonists for PR-B or partial agonists with a stronger agonistic activity on PR-A and a stronger antagonistic activity on PR-B. Other exemplary ligands according to the invention are selective PR-A antagonists and selective PR-B agonists or partial PR-A antagonists/PR-B agonists.

The tissue-selective PR ligands identified through the first and second method for screening for tissue-selective PR ligands according to the second and third aspect of the present invention are preferably selective for breast tissue or uterine tissue. More preferably, they have no impact on PR mediated effects or even inhibit PR mediated effects in breast tissue (such as proliferation/differentiation of the mammary gland), but enhance or maintain PR mediated effects in uterine tissue, i.e. exert a protective, such as an antiproliferative, effect in uterine tissue. Thus, for example, tissue-selective PR ligands and/or PR-isoform specific ligands can be obtained having the desired, beneficial effects in the uterus and/or the ovary without inducing unwanted or even dangerous side-effects in the breast, such as e.g. the induction of proliferation/differentiation of the mammary tissue (which is evident by increased formation of terminal end buds in the mammary glands), which may induce breast cancer. However, as already mentioned above, the benefit of the present invention is not limited to breast/uterine tissue and/or to the progesterone receptor, but can be applied to other target tissues and/or receptor/ligand systems as well.

Preferably, the tissue-selective, preferably breast/uterus-selective PR ligands identified by the second method for screening for tissue selective PR ligands according to the third aspect of the present invention are PR-A or PR-B selective ligands and more preferably, they are pure PR-A agonists, pure PR-B antagonists or partial agonists/antagonists with stronger agonistic effects on PR-A and stronger antagonistic effects on PR-B. Thus, the selected tissue-selective ligands preferably activate or maintain PR-A transactivation in a first tissue and inhibit or do not influence PR-B transactivation in a second tissue.

Due to the beneficial effects of the PR ligands identified through the methods according to the first, second and third aspect of the present invention, these ligands (PR isoform-specific and/or tissue-selective PR ligands) are suitable for use as medicaments, in particular for not affecting or for inhibiting PR mediated effects in a first selected tissue and, at the same time, enhancing or maintaining PR mediated effects in a second target tissue, wherein the first tissue is preferably breast tissue and the second tissue is preferably uterine or ovarian tissue. These medicaments may preferably be used in fertility control (e.g. oral contraception) or HRT. Preferably, the PR ligand identified may be used in inhibiting or not influencing proliferation/differentiation in breast tissue, and in enhancing or maintaining protective, such as antiproliferative, effects in uterine tissue while also maintaining the potentially desired effects on ovulation.

The PR ligands identified through the methods according to the first, second and third aspect of the present invention may also be used for the manufacture of a medicament for not affecting or for inhibiting PR mediated effects (e.g. proliferation/differentiation) in a first target tissue, preferably breast tissue, and enhancing or maintaining beneficial (e.g. antiproliferative) PR mediated effects in a second target tissue, preferably uterine or ovarian tissue (e.g., for use in fertility control or HRT). However, any other related use of the ligands according to the present invention or of the medicaments manufactured from the ligands according to the present invention is envisaged.

Another aspect of the present invention is a method for selectively inhibiting or selectively stimulating PR-A or PR-B in a patient, comprising the step of administering a therapeutically effective amount of a PR isoform A or B specific ligand identified through the method of the present invention to a patient in need thereof.

Another related aspect of the present invention is a method for selectively modulating PR mediated conditions in a selected target tissue comprising the step of administering a therapeutically effective amount of a tissue-selective and/or isoform-specific PR ligand identified through one of the methods of the present invention to a patient in need thereof. Preferably, the modulation of PR mediated conditions comprises inhibiting or not affecting PR mediated, preferably PR-B mediated, effects in a first selected tissue, preferably breast tissue, and enhancing or maintaining PR mediated, preferably PR-A mediated, effects in a second selected tissue, preferably uterine or ovarian tissue. Thus, in the method for selectively modulating PR mediated conditions according to the present invention, preferably protective PR-A mediated effects (such as antiproliferative effects) are enhanced or maintained and potentially dangerous PR-B mediated effects (such as proliferation/differentiation) are inhibited or not influenced. However, also methods wherein PR-A mediated effects are inhibited or not influenced, but PR-B mediated effects are enhanced or maintained, are exemplary methods according to the present invention.

A further aspect of the present invention is an assay kit for screening for PR isoform A or B selective ligands. This kit comprises first cells being stably transfected with a plasmid expressing PR-A and second cells being stably transfected with a plasmid expressing PR-B, said first and second cells being further stably transfected with a plasmid comprising a reporter gene linked to a hormonally responsive promoter, wherein the contacting of said first and second cells with a ligand to be tested yields a level of expressed reporter gene product for said first cells and for said second cells and the comparison of these product levels results in a difference of transcription efficacies and/or potencies induced by said ligand in said first and said second cells, wherein this difference in transcription efficacies and/or potencies is indicative of a selectivity for PR-A or PR-B. Preferably, the cells comprised in the kit are SK-N-MC cells having all the properties as described above for the screening methods according to the first and second aspect of the present invention. Also, the determination of the efficacy and/or potency of the ligand to be tested in the assay kit according to the present invention is performed exactly as described above for the respective screening methods. Again, potential isoform-specific PR ligands identified by means of the assay kit according to the present invention can be of steroidal or non-steroidal nature. The kit is useful for screening for compounds having already been tested on PR affinity/activity in general and found to bind to and activate or inhibit PR, but also with compounds of which their PR ligand potential is unknown. In case the compounds to be tested in the assay have already qualified as PR ligands, they can be pure agonists, pure antagonists or partial agonists/antagonists of PR. Preferably, the isoform-specific PR ligands are pure PR-B antagonists and pure PR-A agonists or partial PR-B antagonists/PR-A agonists and thus exert their beneficial effects in the uterus and/or the ovary whereas unwanted and potentially dangerous side-effects in the breast, e.g., due to differentiation/proliferation of the mammary tissue, are prevented. However, also isoform-specific PR ligands being pure PR-B agonists and pure PR-A antagonists or partial PR-B agonists/PR-A antagonists are in accordance with the present invention.

As already mentioned above, a further aspect of the invention are the SK-N-MC cell lines per se, i.e. the cell lines which are preferably used in the methods according to the first and second aspect of the present invention and the assay kit according to the present invention. A detailed description of the SK-N-MC cell lines according to the present invention has already been given with respect to the screening methods according to the first and second aspects of the present invention. Example 5 gives a detailed description of how the SK-N-MC cell lines according to the present invention may exemplarily be obtained.

The assay method and the kit according to the present invention as presented above are not only suitable for screening for isoform-specific PR ligands, but, with the appropriate variations, may in principle also be applied to other receptor systems. Such further envisaged receptor systems may be other intracellular receptors existing in two or more different isoforms, e.g. the androgen receptor. It is within the purview of the person skilled in the art to perform the necessary alterations to the methods and the kit as described herein to adjust them to screening for isoform-specific and/or tissue-selective ligands of these further envisaged receptors.

The suitability of the method for screening for PR-A or PR-B specific ligands according to the first aspect of the present invention is demonstrated in Example 1 with respect to some exemplary PR ligands. Examples of in vivo tests are provided in Examples 2, 3 and 4. Example 5 pertains to the manufacture of the PR isoform-specific cell lines according to the invention by stable transfections.

### EXAMPLES

### Example 1

Method for Screening for Isoform-Specific PR Ligands

The method for screening for isoform-specific PR ligands according to the present invention is carried out with first and second SK-N-MC cells stably transfected with a plasmid expressing the hPR-A (first cells) or the hPR-B (second cells) and the LUC reporter gene linked to the hormonally responsive MTV promoter. A detailed description of how the cell lines according to the invention and used in this Example may be obtained is given in Example 5.

The cells are cultured in Minimum Essential Medium with Earl's Salts (S-MEM, without L-glutamine; Gibco BRL, no. 21090-022), supplemented with 10% fetal calf serum (FCS), penicillin 100U/streptomycin 100 μg/ml (Biochrom, no. A2213), L-glutamine 4 mmol/l (Gibco BRL, no. 25030-024), sodium pyruvate 1 mmol/l (Biochrom, no. L0473) and 1× non-essential amino acids (Biochrom, no. K0293) at a temperature of 37° C. and in an atmosphere of 5% carbon dioxide.

For the transactivation assay, the cells are seeded onto 96-well dishes (2×10⁴ cells/dish) and cultured in a medium as described above, with the exception that the FCS is replaced by a 3% charcoal stripped FCS. 48 hours later, cells are contacted with prediluted test compounds. For determination of agonistic activity, cells are cultured in the presence of increasing concentrations (10⁻⁶ to 10⁻¹¹ mol/l) of test compounds. As a positive control for reporter gene induction cells are treated with 10⁻⁶ to 10⁻¹¹ mol/l R5020 (promegestone). As a negative control for reporter gene induction, cells are cultured in 1% ethanol. For determination of antagonistic activity, cells are treated with 10⁻¹⁰ mol/l R5020 and additionally with increasing concentrations (10⁻⁶ to 10⁻¹¹ mol/l) of test compounds. As a positive control for inhibition of reporter gene transcription, cells are cultured in increasing concentrations (10⁻⁷ to 10⁻¹² mol/l) of the antiprogestin mifepristone. As a negative control for inhibition of reporter gene transcription, cells are cultured in 1% ethanol.

After incubation with test compounds for 24 hours, the medium is removed and cells are lysed with 20 μl of lysis buffer (Luciferase Assay System E 153A; Promega) and under agitation of the plate for 10 min. After addition of 30 μl of luciferase reagent (Luciferase Assay System E 151A+152A; Promega) within 30 seconds per plate, the activity of the luciferase reporter gene product is determined in the cell lysates by means of a Microlite ML 3000 microtiter plate luminometer (Dynatech) in cycle mode.

Evaluation of the response gives the efficacy [%] for both agonism and antagonism, and evaluation of the EC₅₀ (for agonism) and IC₅₀ (for antagonism) values gives the potency [nM]. Calculation of these values is conducted as follows:

Calculation of Agonistic Activity:

The LUC activity [%] for the measured data points is calculated as follows:

$$relative LUC activity [ \% ] = 100 \times \frac{response {10}^{- 6} to {10}^{- 11} mol / l test compound - CO}{CI - CO}$$

wherein CI=100% stimulation (R5020, 10⁻⁷ mol/l)

and CO=0% stimulation (ethanol, 1%).

Thus, the efficacy [%] is determined according to:

$$efficacy [ \% ] = 100 \times \frac{response {10}^{- 7} mol / l test compound - CO}{CI - CO}$$

The potency [nM], i.e. the EC₅₀, is determined graphically.

Calculation of Antagonistic Activity:

The LUC activity [%] for the measured data points is calculated as follows:

$$relative LUC activity [ \% ] = 100 \times \frac{response {10}^{- 6} to {10}^{- 12} mol / l test compound - CO}{CI - CO}$$

wherein CI=0% inhibition (R5020, 10⁻¹⁰ mol/l) (corresponds to 100% stimulation)

and CO=100% inhibition (EtOH, 1%) (corresponds to 0% stimulation)

Thus, the efficacy [%] is determined according to:

$$efficacy [ \% ] = 100 - ( 100 \times \frac{response {10}^{- 7} mol / l test compound - CO}{CI - CO} )$$

The potency [nM], i.e. the IC₅₀, is determined graphically.

Thus, the efficacy [%] and the potency [nM] for each test ligand can be calculated. Some efficacy results achieved for different test ligands are presented below.

As explained above, the selectivity of the tested ligands for one of the PR isoforms, A or B, is defined as the difference in transcription efficacies achieved for PR-A and PR-B, which must be above or equal to 10%, preferably above or equal to 15% and more preferably even higher, such as above or equal to 20%, and/or the difference in potency achieved for such ligands, which must be of a factor of at least 10.

Results:

Pure Agonists with Selectivity for PR-A:

a) 13-ethyl-9,11-dihydro-17-hydroxy-(11α,17α)-6′H-benzo[10,9,11]-18,19-dinorpregna-4,9(11),20-trien-3-one (disclosed in WO 95/27725 and WO 94/09025):

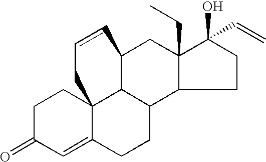

Agonism efficacy (PR-A): 132%; antagonism efficacy (PR-A): none.

Agonism efficacy (PR-B): 108%; antagonism efficacy (PR-B): none.

Difference of agonism efficacies (PR-A —PR-B): 24% (indicating PR-A selectivity).

b) 13-ethyl-9,11-dihydro-17-hydroxy-(11α,17α)-6′H-benzo[10,9,11]-18,19-dinorpregna-4,9(11)-dien-20-yn-3-one:

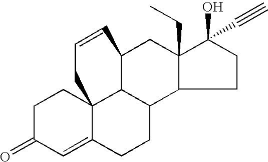

Agonism efficacy (PR-A): 129%; antagonism efficacy (PR-A): none.

Agonism efficacy (PR-B): 108%; antagonism efficacy (PR-B): none.

Difference of agonism efficacies (PR-A —PR-B): 21% (indicating PR-A selectivity)

c) 9,11-dihydro-17-hydroxy-(11α,17α)-6′H-benzo[10,9,11]-19-norpregna-4,9(11), 15-trien-20-yn-3-one (disclosed in WO 95/27725 and WO 94/09025):

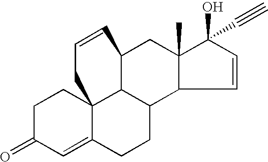

Agonism efficacy (PR-A): 131%; antagonism efficacy (PR-A): none.

Agonism efficacy (PR-B): 114%; antagonism efficacy (PR-B): none.

Difference of agonism efficacies (PR-A —PR-B): 17% (indicating PR-A selectivity).

Pure Agonists with Selectivity for PR-B:

a) 17-acetoxy-6-methyl-4,6-pregnadiene-3,20-dione:

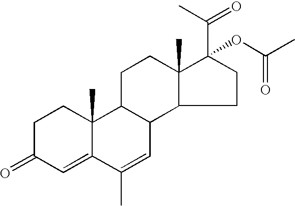

Agonism efficacy (PR-A): 35%; antagonism efficacy (PR-A): none.

Agonism efficacy (PR-B): 81%; antagonism efficacy (PR-B): none.

Difference of agonism efficacies (PR-B−PR-A): 46% (indicating PR-B selectivity).

b) 17-(acetyloxy)-9,11-dihydro-6-methyl-(6α,11α)-6H′benzo[10,9,11]-19-norpregna-4,9(11)-diene-3,20-dione (disclosed in WO 95/27725 and WO 94/09025):

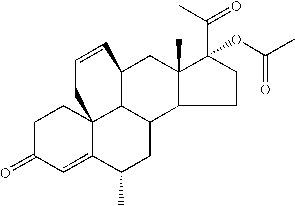

Agonism efficacy (PR-A): 50%; antagonism efficacy (PR-A): none.

Agonism efficacy (PR-B): 90%; antagonism efficacy (PR-B): none.

Difference of agonism efficacies (PR-B−PR-A): 40% (indicating PR-B selectivity).

c) 1-(3-bromophenyl)-N-(1,3-dihydro-1-oxo-5-isobenzofuranyl)-α-hydroxy-α-(trifluoromethyl)-cyclopropanepropanamide (disclosed in WO 98/54159):

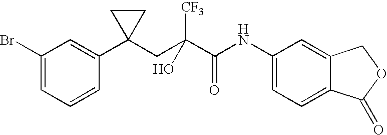

Agonism efficacy (PR-A): 81%; antagonism efficacy (PR-A): none.

Agonism efficacy (PR-B): 106%; antagonism efficacy (PR-B): none.

Difference of agonism efficacies (PR-B−PR-A): 25% (indicating PR-B selectivity).

d) 13-ethyl-5α,9-dihydro-17-hydroxy-(17α)-6′H-benzo[10,9,11]-18,19-dinorpregna-4,9(11),15-trien-20-yn-3-one (disclosed in WO 94/09025):

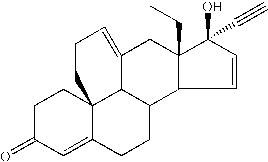

Agonism efficacy (PR-A): 85%; antagonism efficacy (PR-A): none.

Agonism efficacy (PR-B): 108%; antagonism efficacy (PR-B): none.

Difference of agonism efficacies (PR-B−PR-A): 23% (indicating PR-B selectivity).

e) 5-{[2-hydroxy-4-phenyl-2-(trifluoromethyl)pentyl]amino}-1(3H)-isobenzofiuranone (disclosed in WO 98/54159):

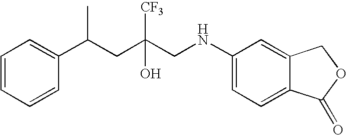

Agonism efficacy (PR-A): 39.9%; antagonism efficacy (PR-A): none.

Agonism efficacy (PR-B): 56.8%; antagonism efficacy (PR-B): none.

Difference of agonism efficacies (PR-B−PR-A): 17% (indicating PR-B selectivity).

Partial Agonists with Selectivity (Agonism) for PR-B and Selectivity (Antagonism) for PR-A:

a) α-hydroxy-N-[4-nitro-3-(trifluoromethyl)phenyl]-o-(trifluoromethyl)-benzenebutanamide (disclosed in EP 253 503):

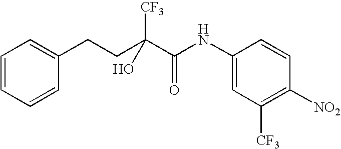

Agonism efficacy (PR-A): 18%; antagonism efficacy (PR-A): 59%.

Agonism efficacy (PR-B): 59%; antagonism efficacy (PR-B): 36%.

Difference of agonism efficacies (PR-B−PR-A): 41% (indicating PR-B selectivity).

Difference of antagonism efficacies (PR-A−PR-B): 23% (indicating PR-A selectivity).

b) 17-(acetyloxy)-6-chloro-9,11-dihydro-(11α)-cyclopenta[10,9,11]-19-norpregna-4,6,9(11)-triene-3,20-dione (disclosed in WO 95/11915):

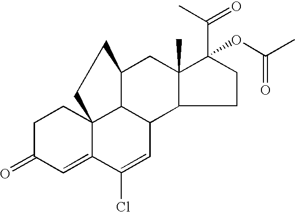

Agonism efficacy (PR-A): 29%; antagonism efficacy (PR-A): 45%.

Agonism efficacy (PR-B): 50%; antagonism efficacy (PR-B): 30%.

Difference of agonism efficacies (PR-B−PR-A): 21% (indicating PR-B selectivity).

Difference of antagonism efficacies (PR-A —PR-B): 15% (indicating PR-A selectivity).

### Example 2

Bioassay on Proliferatin/Differentiating Effects in the Rat Mammary Epithelium

The object of this test is to evaluate the effect of progestins (PR ligands) on the development of the mammary gland, in particular on the formation of terminal end buds in the mammary gland in estrogen primed rats.

Premature female rats (Wistar Han, SPF) are ovariectomized at the age of 21 days, 4 to 6 days before treatment start. The animals are treated for 6 days with standard estrogen (estrone, 70 μg/kg) and the test ligand (application volume: 0.1 ml/50 g body weight; vehicle: benzylbenzoate/castor oil (1+4 v/v); subcutaneous). Control groups are e.g.: vehicle, estradiol without progestin, estradiol together with a known progestin, e.g. R5020. After the 6 day treatment the animals are killed with carbon dioxide.

For the whole mount staining, animals are shaved in the left abdominal inguinal mammary region, which is cut from the body together with the skin. For the histological/immunohistochemical analyses the right abdominal inguinal mammary gland is cut from the body together with the connective tissue adhered thereto and fixed in 3.7% formalin in PBS (phosphate buffer saline; without Ca²⁺/Mg²⁺).

Whole Mount Staining.

The preparations are fixed over night in alcohol-formalin according to the method of Tellyesniczky (see below). Then the mammary gland tissue and subcutis adhered thereto are stripped from the cutis and the preparations are again fixed over night. The further steps are as follows: ethanol 70%: 1.5 hours; acetone: 3×1.5 hours; acetone: over night; isopropanol: 1.5 hours; ethanol 96%: 2 hours; hematoxylin-iron: 3 hours; VE water: first rinse the preparations and then 2×0.5 hours; ethanol 70%: over night; ethanol 80%: 1.5 hours; ethanol 96%: 1.5 hours; isopropanol: 1.5 hours. The preparations are then moved to petri dishes and left in toluene for approximately 1 hour, i.e. until they have stopped to swim up. Then the preparations are treated with cedarwood oil (Merck, no. 1.06965). The incubation times above are minimum times and can be extended. In particular, incubation in ethanol 70% after fixation can be extended to at least 2.5 weeks.

Preparation of the Solutions Necessary for the Whole Mount Staining:

a) Alcohol-formalin according to Tellyesniczky: formaldehyde 37%: 81.8 ml, ethanol 70%: 1636 ml, glacial acetic acid (to be added shortly before use): 81.8 ml (total: 1800 ml).

b) Hematoxylin mother solution: Hematoxylin (Merck, no. 1.15938): 10 g, ethanol 96%: 100 ml. The solution must stand for 48 hours at 37° C. before use. It can be kept in a dark place for almost unlimited time.

c) Hematoxylin-iron solution for use: hematoxylin mother solution (filtered): 15.2 ml, ethanol 96%: 1374 ml, FeCl₃×6H₂O (s. 4): 91.1 ml, 1 mol/l HCl: 220 ml (total: 1700 ml); adjustment to a pH of 1.25 with 2 mol/l NaOH.

d) FeCl₃×6H₂O solution: FeCl₃×6H₂O (Merck, no. 1.03943): 1.07 g, VE water: 90.2 ml, HCl: 37%: 0.92 ml (total: 91.1 ml).

By means of a 40-fold magnification, the terminal end buds near the nipple in direction of the tail are counted. The area to be investigated should be about 1.8 mm². For well-differentiated preparations this area may be reduced, with at least 250 buds to be counted. After counting, the number of end buds per 1 mm² is calculated.

Evaluation:

The progestagenic effect of a test compound is either determined as a threshold value (concentration at which a significant progestagenic effect is recognized for the first time), or the dose that is equi-efficient to 0.3 mg/kg per day R5020 is determined.

MIB-5 Immunohistochemistry (According to C. Gerlach et al., Lab. Invest. 1997, 77(6), 697-698, with Modifications):

Mammary glands are fixed in 4% formaldehyde/PBS for 24 h and embedded in paraffin. 4 μm sections are spread on slides, deparaffinized, treated with microwaves for 10 min. in citrate buffer pH 6.0 and rinsed with PBS. Slides are then blocked with 3% H₂O₂/methanol for 15 min., Blockingkit (Vektor, no. SP-2001) for 10 min. and rat serum (Sigma, no. S-7648) diluted 1:2 in PBS for 30 min to reduce nonspecific staining and rinsed in PBS. Slides are incubated for 1 hour with monoclonal antibody MIB-5 (Dianova, no. Dia-5055), which is specific for the rat Ki-67 antigen (1:200 diluted in PBS/0.2% BSA). Then, slides are washed twice in PBS/0.2% TWEEN 20, incubated with biotinylated rat anti-mouse secondary antibodies (Dianova, no. 425-066-100), diluted 1:200 in PBS/0.2% TWEEN 20 for 1 hour and washed again twice in PBS/0.2% TWEEN 20, following an incubation with avidin-biotin-peroxidase complexes (Vecstain Elite ABC Kit no. PK-6100) for 1 h. Staining is performed by means of diaminobenzidine (Zymed Substrate Kit). All steps are performed at room temperature.

Evaluation:

In order to characterize a test compound, the percentage of MIB-5 stained mammary epithelium cells is determined.

### Example 3

Pregnancy Maintenance Test in Rat

In rats, castration induces termination of pregnancy. Progestins (combined with estrogens) are capable of maintaining pregnancy in castrated animals. However, the degree of pregnancy maintenance in castrated rats is optimal only in a defined dose range. Therefore, higher as well as lower doses generally induce a weaker effect. Accompanying treatment with defined doses of estrone (E₁) increases the pregnancy maintaining effect of progestins.

Pregnant rats (Wistar Han, SPF) of 190 to 220 g (5 to 8 animals per dose) are ovariectomized on day 8 of pregnancy, 2 hours after the first substance administration. From day 8 to day 14, rats are daily treated with test compound in combination with a standard dose of E₁. One day later, animals are killed with carbon dioxide. For each animal, the number of living and dead fetuses is determined according to the heartbeat of the embryos. In case of empty uteri, the number of implantation sites is determined by means of staining with a 10% ammonium sulfide solution.

Formulation and Application of Test Compounds and Estrone:

S.c. (subcutaneous) application: The test compound is dissolved in benzyl benzoate/castor oil (1+4 v/v) and the daily dose is administered in a volume of 1 ml/kg body weight.

P.o. (peroral) application: The test compound is suspended in a carrier liquid (85 mg Myrj^{R} in 100 ml 0.9% w/v NaCl solution) and the daily dose is administered in a volume of 2 ml/kg body weight.

I.p. (intraperitoneal) application: The test compound is dissolved in propylene glycol and charged in miniature osmotic pumps (type 2001, 1.0 μl/h, 7 days), which are placed in the abdominal cavity of the rat

The standard dose of estrone is 0.005 mg/kg body weight s.c. and is dissolved in benzyl benzoate/castor oil (1.4 v/v).

Evaluation:

It is determined the pregnancy maintenance per animal [%], the pregnancy maintenance per dose (median of single salues) and the ED₅₀ (dose, at which pregnancy is maintained in 50% of the animals; 100% corresponds to control animals that are not ovariectomized).

### Example 4

Ovulation Inhibition Test in Rat

Before the treatment is started, two menstrual cycles of female rats (Wistar Han, SPF; weight: 190 to 210 g) are monitored. Only animals having a regular 4 day-cycle are used for the subsequent test. Starting in the metoestrus, the test compound is administered for 4 days (day 1 to 4) and the cycle is controlled thereafter.

For subcutaneous application, the test compounds are dissolved in benzyl benzoate/castor oil (1+9 v/v) and the daily dose is administered in a volume of 1 ml/kg body weight.

For peroral application, the test compounds are suspended in a carrier liquid (85 mg Myrj^{R} in 100 ml 0.9% w/v NaCl solution) and the daily dose is administered in a volume of 2 ml/kg body weight.

On day 4, after the application of the test compound, those animals having estrus or metoestrus are ovariectomized under ether anesthesia on one side. Preparations of the tubes are prepared and they are investigated for ova by means of microscope. On day 5, all animals (intact and partly ovariectomized) are killed with carbon dioxide, the tubes are preserved and analyzed in the same way.

Evaluation:

It is determined, as a percentage, in how many animals ovulation was inhibited.

### Example 5

Stable Transfections

The stable transfections were performed using Lipofectamine Reagent (Gibco BRL, Berlin). SK-N-MC cells were seeded onto 6-well dishes at a density of 5×10⁵ cells per dish. Cells were typically about 80% confluent after 24 hours. Before transfection, cells were washed twice with 1 ml Opti-MEM (Gibco, BRL, Berlin) per dish. For each dish, 5 μg DNA (pRSVhPR-neo: PR-B plasmid) or 10 μg DNA (5 μg phPR-2: PR-A plasmid and 5 μg pRSV-neo), respectively, were diluted with 500 μl Opti-MEM. Next, the DNA and Lipofectamine Reagent dilutions were combined in a polystyrene snap cap tube to obtain 1 ml of transfection solution per dish, gently mixed, incubated at room temperature for 45 min. and added to the washed cells. After 5 hours, the transfection solution was replaced by 2 ml S-MEM supplemented with 10% FCS. After 48 hours, the cells were trypsinized and replated onto 100 dishes at a density of 3×10⁴ and 1.5×10⁴, respectively. SK-N-MC cell clones either stably transfected with pRSVhPR-neo or phPR-2 and pRSV-neo were selected in medium supplemented with 800 μg G418. Neomycin-resistant clones were stably transfected with pMTV-LUC and pSV2pac and were selected in culture medium supplemented with 400 mg/ml G418 and 0.2 mg/ml puromycin. Finally, clone SK-N-MC C 23.43 (stably expressing PR-A and MTV-LUC) and clone SK-N-MC VIII 1.1 (stably expressing PR-B and MTV-LUC) were selected for transactivation studies.

The plasmids for the stable transfections described above were obtained as follows:

For PR-A:

phPR-2: According to Kastner et al., J. Biol. Chem. 1990, 265, 12163-12167.

pMTV-LUC: From A. Cato, Intitut für Genetik, Kemforschungszentrum Karlsruhe.

pRSV-neo:

a) Construction of PRSV-CAT from SV2-CAT (see Gorman et al., Proc. Natl. Acad. Sci. 1982, 79, 6777-6781).

b) Construction of pRSV-neo from RSV-CAT (see Gorman et al., Science 1983, 221, 551-555).

pSV2pac: According to P. Artelt, Gene 1988, 68, 213-219.

For PR-B:

pRSV hPR-neo: Contruct from pRC/RSV (containing the neomycin resistance gene expressed from SV40 early promoter; from Invitrogen, San Diego) and hPR-1 (see Kastner et al., J. Biol. Chem. 1990, 265, 12163-12167).

The vector pRC/RSV is digested with Not I and Xba I; for the insert, hPR-1 digested with EcoR 1, linker ligation with Not 1, restriction digestion with Not I and Xba I resulting in 2.8 Kb fragment, which is isolated and ligated into the above vector.

pMTV-LUC: From A. Cato, Institut für Genetik, Kernforschungszentrum Karlsruhe.

pMPSVEH: According to P. Artelt, Gene 1988, 68, 213-219.

1. A method for screening for progesterone receptor isoform A or B specific ligands, comprising the steps of
(a) stably transfecting first cells with a plasmid expressing the progesterone receptor isoform A;
(b) stably transfecting second cells with a plasmid expressing the progesterone receptor isoform B;
(c) further stably transfecting said first and said second cells with a plasmid comprising a reporter gene linked to a hormonally responsive promoter;
(d) contacting said first and said second cells with a ligand to be tested;
(e) determining the transcription efficacy and/or potency of said reporter gene in said first and second cells; and
(f) selecting a ligand having a selectivity for the progesterone receptor isoform A or the progesterone receptor isoform B.

2. The method according to claim 1, wherein the selectivity in step (f) is defined such that the difference in transcription efficacy determined for said ligand in said first and said second cells is above or equal to 10% and/or the potency achieved with said ligand in said first cells differs by a factor of at least 10 from the potency achieved with said ligand in said second cells.

3. The method according to claim 1, wherein the cells are SK-N-MC cells.

4. The method according to claim 1, wherein the hormonally responsive promoter is the mouse mammary tumor virus (MMTV) promoter.

5. The method according to claim 1, wherein the reporter gene is the luciferase (LUC) reporter gene.

6. The method according to claim 5, wherein the transcription efficacy and/or potency in said first and said seconds cells is determined by measuring the luciferase activity.

7. The method according to claim 1, wherein the plasmid expressing the progesterone receptor isoform A or the progesterone receptor isoform B and the plasmid comprising the promoter and reporter gene comprise an antibiotic resistance gene.

8. The method according to claim 7, wherein the antibiotic resistance gene is a neomycin or puromycin resistance gene.

9. The method according to claim 8, wherein the plasmid expressing the progesterone receptor isoform A or the progesterone receptor isoform B comprises a neomycin resistance gene and the plasmid comprising the reporter gene linked to the hormonally responsive promoter comprises a puromycin resistance gene.

10. A method for screening for tissue-selective progesterone receptor ligands, comprising the steps of
(a) stably transfecting first cells with a plasmid expressing the progesterone receptor isoform A;
(b) stably transfecting second cells with a plasmid expressing the progesterone receptor isoform B;
(c) further stably transfecting said first and said second cells with a plasmid comprising a reporter gene linked to a hormonally responsive promoter;
(d) contacting said first and said second cells with a ligand to be tested;
(e) determining the transcription efficacy and/or potency of said reporter gene in said first and second cells;
(f) selecting a ligand having a selectivity for the progesterone receptor isoform A or the progesterone receptor isoform B;
(g) subjecting the ligand selected in step (f) to in vivo tests in a first and a second target tissue; and
(h) selecting the ligand having the desired activity with respect to said first and said second target tissue.

11. The method according to claim 10, wherein the first tissue is breast tissue and the second tissue is uterine tissue.

12. The method according to claim 10, wherein the in vivo test is a test to determine progesterone-mediated effects in said first and said second tissue.

13. The method according to claim 10, wherein the selectivity in step (f) is defined such that the difference in transcription efficacy determined for said ligand in said first and said second cells is above or equal to 10% and/or the potency achieved with said ligand in said first cells differs by a factor of at least 10 from the potency achieved with said ligand in said second cells.

14. The method according to claim 10, wherein the cells are SK-N-MC cells.

15. The method according to claim 10, wherein the hormonally responsive promoter is the mouse mammary tumor virus (MMTV) promoter.

16. The method according to claim 10, wherein the reporter gene is the luciferase (LUC) reporter gene.

17. The method according to claim 16, wherein the transcription efficacy and/or potency in said first and said seconds cells is determined by measuring the luciferase activity.

18. The method according to claim 10, wherein the plasmid expressing the progesterone receptor isoform A or the progesterone receptor isoform B and the plasmid comprising the promoter and reporter gene comprise an antibiotic resistance gene.

19. The method according to claim 18, wherein the antibiotic resistance gene is a neomycin or puromycin resistance gene.

20. The method according to claim 19, wherein the plasmid expressing the progesterone receptor isoform A or the progesterone receptor isoform B comprises a neomycin resistance gene and the plasmid comprising the reporter gene linked to the hormonally responsive promoter comprises a puromycin resistance gene.

21. A progesterone receptor isoform A or B specific ligand, identified through the method of claim 1.

22. The progesterone receptor isoform A or B specific ligand according to claim 21, wherein the progesterone receptor isoform A or B specific ligand is a pure agonist, pure antagonist or partial agonist/antagonist of the progesterone receptor isoform A or the progesterone receptor isoform B.

23. The progesterone receptor isoform A or B specific ligand according to claim 22, wherein the progesterone receptor isoform A or B specific ligand is a progesterone receptor isoform A selective agonist or a progesterone receptor isoform B selective antagonist or a partial progesterone receptor isoform A agonist and progesterone receptor isoform B antagonist.

24. A tissue-selective progesterone receptor ligand, identified through the method of claim 10.

25. The tissue-selective progesterone receptor ligand according to claim 24, wherein the tissue-selective progesterone receptor ligand is selective for breast tissue or uterine tissue.

26. The tissue-selective progesterone receptor ligand according to claim 25, wherein the tissue-selective progesterone receptor ligand does not influence or inhibits progesterone receptor mediated effects in breast tissue and enhances or maintains progesterone-receptor mediated effects in uterine tissue.

27. A SK-N-MC cell line stably transfected with a plasmid expressing the progesterone receptor isoform A or the progesterone receptor isoform B.

28. The SK-N-MC cell line according to claim 27, further stably transfected with a plasmid comprising a reporter gene linked to a hormonally responsive promoter.

29. The SK-N-MC cell line according to claim 28, wherein the plasmid expressing the progesterone receptor isoform A or the progesterone receptor isoform B and the plasmid comprising the promoter and reporter gene comprise an antibiotic resistance gene.

30. The SK-N-MC cell line according to claim 29, wherein the antibiotic resistance gene is a neomycin or puromycin resistance gene.

31. The SK-N-MC cell line according to claim 30, wherein the plasmid expressing the progesterone receptor isoform A or the progesterone receptor isoform B comprises a neomycin resistance gene and the plasmid comprising the reporter gene linked to the hormonally responsive promoter comprises a puromycin resistance gene.

32. The SK-N-MC cell line according to claim 28, wherein the reporter gene is a luciferase (LUC) reporter gene and the hormonally responsive promoter is the mouse mammary tumor virus (MMTV) promoter.

33. An assay kit to screen for progesterone receptor isoform A or B selective ligands, comprising first cells being stably transfected with a plasmid expressing the progesterone receptor isoform A and second cells being stably transfected with a plasmid expressing the progesterone receptor isoform B, said first and second cells being further stably transfected with a plasmid comprising a reporter gene linked to a hormonally responsive promoter, wherein the contacting of said first and second cells with a ligand to be tested yields a level of expressed reporter gene product for said first cells and for said second cells and the comparison of these product levels results in a difference in transcription efficacy and/or potency induced by said ligand in said first and said second cells wherein this difference in transcription efficacy is indicative of a selectivity for the progesterone receptor isoform A or progesterone receptor isoform B.

34. The assay kit according to claim 33, wherein the difference in transcription efficacy determined for said ligand in said first and said second cells is above or equal to 10% and/or the potency achieved with said ligand in said first cells differs by a factor of at least 10 from the potency achieved with said ligand in said second cells.

35. The assay kit according to claim 33, wherein the cells are SK-N-MC cells.

36. The assay kit according to claim 33, wherein the hormonally responsive promoter is the mouse mammary tumor virus (MMTV) promoter.

37. The assay kit according to claim 33, wherein the reporter gene is the luciferase (LUC) reporter gene.

38. The assay kit according to claim 37, wherein the transcription efficacy and/or potency in said first and said second cells is determined by measuring the luciferase activity.

39. The assay kit according to claim 33, wherein the plasmid expressing the progesterone receptor isoform A or the progesterone receptor isoform B and the plasmid comprising the promoter and reporter gene comprise an antibiotic resistance gene.

40. The assay kit according to claim 39, wherein the antibiotic resistance gene is a neomycin or puromycin resistance gene.

41. The assay kit according to claim 40, wherein the plasmid expressing the progesterone receptor isoform A or the progesterone receptor isoform B comprises a neomycin resistance gene and the plasmid comprising the reporter gene linked to the hormonally responsive promoter comprises a puromycin resistance gene.

42. A progesterone receptor isoform A or B specific ligand identified through the method as defined in claim 1 for use as a medicament.

43. The progesterone receptor isoform A or B specific ligand according to claim 42 for use in selectively inhibiting or stimulating progesterone receptor isoform A or progesterone receptor isoform B mediated effects.

44. The progesterone receptor isoform A or B specific ligand according to claim 43 for use in not influencing or selectively inhibiting progesterone receptor isoform B and selectively enhancing or maintaining progesterone receptor isoform A mediated effects.

45. The progesterone receptor isoform A or B specific ligand according to claim 42 for use in not influencing or inhibiting progesterone receptor mediated effects in a first selected tissue and, at the same time, enhancing or maintaining progesterone receptor mediated effects in a second selected tissue.

46. The progesterone receptor isoform A or B specific ligand according to claim 42 for use in fertility control or hormone replacement therapy.

47. The progesterone receptor isoform A or B specific ligand according to claim 45, wherein the first selected tissue is breast tissue and the second selected tissue is uterine tissue.

48. The progesterone receptor isoform A or B specific ligand according to claim 45 for use in selectively inhibiting or not influencing proliferation and differentiation of breast tissue and/or selectively enhancing or maintaining antiproliferative effects in uterine tissue.

49. Use of a progesterone receptor isoform A or B specific ligand identified through the method of claim 1 for the manufacture of a medicament for selectively inhibiting or stimulating progesterone receptor isoform A or progesterone receptor isoform B mediated effects.

50. The use according to claim 49 for not influencing or inhibiting progesterone receptor mediated effects in a first selected tissue and, at the same time, enhancing or maintaining progesterone receptor mediated effects in a second selected tissue.

51. The use according to claim 49, wherein the medicament is used in fertility control or hormone replacement therapy.

52. The use according to claim 50, wherein the first selected tissue is breast tissue and the second selected tissue is uterine tissue.

53. The use according to claim 49 for selectively inhibiting or not influencing proliferation and differentiation of breast tissue and/or selectively enhancing or maintaining antiproliferative effects in uterine tissue

54. A tissue-selective progesterone receptor ligand identified through the method as defined in claim 10 for use as a medicament.

55. The tissue-selective progesterone receptor ligand according to claim 54 for use in not influencing or inhibiting progesterone receptor mediated effects in a first selected tissue and, at the same time, enhancing or maintaining progesterone receptor mediated effects in a second selected tissue.

56. The tissue-selective progesterone receptor ligand according to claim 54 for use in fertility control or hormone replacement therapy.

57. The tissue-selective progesterone receptor ligand according to claim 55, wherein the first selected tissue is breast tissue and the second selected tissue is uterine tissue.

58. The tissue-selective progesterone receptor ligand according to claim 54 for use in selectively inhibiting or not influencing proliferation and differentiation of breast tissue and/or selectively enhancing or maintaining antiproliferative effects in uterine tissue.

59. Use of a tissue-selective progesterone receptor ligand identified through the method as defined in claim 10, for the manufacture of a medicament for selectively modulating progesterone receptor mediated effects in a first and a second selected tissue.

60. The use according to claim 59 for not influencing or inhibiting progesterone receptor mediated effects in a first selected tissue and, at the same time, enhancing or maintaining progesterone receptor mediated effects in a second selected tissue.

61. The use according to claim 59, wherein the medicament is used in fertility control or hormone replacement therapy.

62. The use according to claim 59, wherein the first selected tissue is breast tissue and the second selected tissue is uterine tissue.

63. The use according to claim 59 for selectively inhibiting or not influencing proliferation and differentiation of breast tissue and/or selectively enhancing or maintaining antiproliferative effects in uterine tissue.

64. A method for selectively inhibiting or stimulating progesterone receptor isoform A or progesterone receptor isoform B mediated effects, comprising the step of administering a therapeutically effective amount of a progesterone receptor isoform A or B specific ligand identified through the method as defined in claim 1 to a patient in need thereof.

65. A method for selectively modulating progesterone receptor mediated conditions in a selected tissue, comprising the step of administering a therapeutically effective amount of a tissue-selective progesterone receptor ligand identified by means of the method as defined in claim 10 to a patient in need thereof.

66. The method according to claim 65, wherein the modulation of progesterone receptor mediated conditions comprises not influencing or inhibiting progesterone receptor mediated effects in a first selected tissue and, at the same time, enhancing or maintaining progesterone receptor mediated effects in a second selected tissue.

67. The method according to claim 66, wherein the first selected tissue is breast tissue and the second selected tissue is uterine tissue.

68. The method according to claim 66, comprising selectively inhibiting or not influencing proliferation and differentiation of breast tissue and/or selectively enhancing or maintaining antiproliferative effects in uterine tissue.

69. A method for screening for tissue-selective progesterone receptor ligands, comprising the steps of
(a) subjecting a progesterone receptor ligand to at least one in vivo test in a first selected tissue,
(b) subjecting said ligand to at least one in vivo test in a second selected tissue, and
(c) selecting a progesterone receptor ligand that selectively modulates progesterone receptor mediated effects in said first selected tissue with respect to said second selected tissue.

70. The method according to claim 69, wherein the progesterone receptor ligand is selected from the group consisting of progesterone receptor pure agonists, pure antagonists and partial agonists/antagonists.

71. The method according to claim 69, wherein the progesterone receptor ligand selected in step (c) inhibits or does not influence progesterone receptor mediated effects in said first selected tissue and enhances or maintains progesterone receptor mediated effects in said second selected tissue.

72. The method according to claim 69, wherein said first selected tissue is breast tissue and said second selected tissue is uterine tissue.

73. The method according to claim 69, wherein the progesterone receptor ligand selected in step (c) selectively inhibits or does not influence proliferation and differentiation in breast tissue and/or selectively enhances or maintains antiproliferative effects in uterine tissue.

74. A tissue-selective progesterone receptor ligand identified through the method of claim 69.

75. The tissue-selective progesterone receptor ligand according to claim 74, selected from the group consisting of progesterone receptor pure agonists, pure antagonists and partial agonists/antagonists.

76. The tissue-selective progesterone receptor ligand according to claim 74, wherein the progesterone receptor ligand inhibits or does not influence progesterone receptor mediated effects in said first selected tissue and enhances or maintains progesterone receptor mediated effects in said second selected tissue.

77. The tissue-selective progesterone receptor ligand according to claim 76, wherein said first selected tissue is breast tissue and said second selected tissue is uterine tissue.

78. The tissue-selective progesterone receptor ligand according to claim 74, wherein said ligand selectively inhibits or does not influence proliferation and differentiation in breast tissue and/or selectively enhances or maintains antiproliferative effects in uterine tissue.

79. The tissue-selective progesterone receptor ligand according to claim 74, selectively/activating or maintaining progesterone receptor isoform A transcription and selectively inhibiting or not influencing progesterone receptor isoform B transcription.

80. A tissue-selective progesterone receptor ligand identified through the method of claim 69 for use as a medicament.

81. The tissue-selective progesterone receptor ligand according to claim 80, selected from the group consisting of progesterone receptor pure agonists, pure antagonists and partial agonists/antagonists.

82. The tissue-selective progesterone receptor ligand according to claim 80, for use in selectively modulating progesterone receptor mediated effects in said first selected tissue with respect to said second selected tissue.

83. The tissue-selective progesterone receptor ligand according to claim 82, for use in inhibiting or not influencing progesterone receptor mediated effects in said first selected tissue and enhancing or maintaining progesterone receptor mediated effects in said second selected tissue.

84. The tissue-selective progesterone receptor ligand according to claim 83, wherein the first selected tissue is breast tissue and the second selected tissue is uterine tissue.

85. The tissue-selective progesterone receptor ligand according to claim 80, wherein said ligand selectively inhibits or does not influence proliferation and differentiation in breast tissue and/or selectively enhances or maintains antiproliferative effects in uterine tissue.

86. The tissue-selective progesterone receptor ligand according to claim 80, selectively activating or maintaining PR-A transcription and selectively inhibiting or not influencing PR-B transcription.

87. Use of a tissue-selective progesterone receptor ligand identified through the method of claim 69 for the manufacture of a medicament for selectively modulating progesterone receptor mediated effects in a first selected tissue with respect to a second selected tissue.

88. The use according to claim 87 for inhibiting or not influencing progesterone receptor mediated effects in said first selected tissue and enhancing or maintaining progesterone receptor mediated effects in said second selected tissue.

89. The use according to claim 88, wherein said first selected tissue is breast tissue and said second selected tissue is uterine tissue.

90. The use according to claim 87 for selectively inhibiting or not influencing proliferation and differentiation of breast tissue and/or selectively enhancing or maintaining antiproliferative effects in uterine tissue.

91. The use according to claim 87, wherein the medicament is used in fertility control or hormone replacement therapy.

92. The use according to claim 87, wherein the tissue-selective progesterone receptor ligand is selected from the group consisting of progesterone receptor pure agonists, pure antagonists and partial agonists/antagonists.

93. The use according to claim 87, wherein the tissue-selective progesterone receptor ligand selectively activates PR-A transcription and selectively inhibits or does not influence PR-B transcription.

94. A method for selectively modulating progesterone receptor mediated effects in a first selected tissue with respect to a second selected tissue, comprising the step of administering a therapeutically effective amount of a tissue-selective progesterone receptor ligand identified through the method of claim 69 to a patient in need thereof.

95. The method according to claim 94 for inhibiting or not influencing progesterone receptor mediated effects in said first selected tissue and enhancing or maintaining progesterone receptor mediated effects in said second selected tissue.

96. The method according to claim 94, wherein said first selected tissue is breast tissue and said second selected tissue is uterine tissue.

97. The method according to claim 94, wherein the tissue-selective progesterone receptor ligand is selected from the group consisting of progesterone receptor pure agonists, pure antagonists and partial agonists/antagonists.

98. The method according to claim 94, wherein the tissue-selective progesterone receptor ligand selectively activates or maintains PR-A transcription and selectively inhibits or does not influence PR-B transcription.

99. The method according to claim 94, comprising selectively inhibiting or not influencing proliferation and differentiation of breast tissue and/or selectively enhancing or maintaining antiproliferative effects in uterine tissue.

100. Pharmaceutical composition, comprising a tissue-selective progesterone receptor ligand identified through the method according to claim 69.
